(19) United States
(12) Patent Application Publication
(10) Pub. No.: US 2011/0048440 A1
(43) Pub. Date: Mar. 3, 2011

(54) Composition of matter comprising a semi-permanent mascara and process to professionally apply the same

(76) Inventor: Jennifer Green, Phoenix, AZ (US)

(21) Appl. No.: 12/539,611

(22) Filed: Aug. 12, 2009

Related U.S. Application Data
(60) Provisional application No. 61/188,656, filed on Aug. 12, 2008.

(51) Int. Cl. A45D 40/26; A61K 8/81; A61Q 1/10

(52) U.S. Cl. 132/200; 424/70.7

(57) ABSTRACT

A composition of matter which includes a type of black pigmented, pharmaceutical grade cyanoacrylate, mixed with a small amount of irregular shaped, insoluble polytetrafluoroethene in microfiber powder form, and then professionally applied to the eyelashes creating a semipermanent mascara application that is 100% waterproof, will not run, smear, or smudge, and lasts 2 to 3 weeks.

### CROSS REFERENCE TO RELATED APPLICATIONS

This application is based on provisional application serial number 61188656, filed on Aug. 12, 2008.

### BACKGROUND OF THE INVENTION

This invention relates generally to the field of cosmetics and more specifically to a composition of matter comprising a semi-permanent mascara that is waterproof, will not run smudge or smear, and lasts 2 to 3 weeks; and the process to professionally apply the same.

Mascara was invented many years ago, and throughout the years, different chemicals and additives have been created in an attempt to make the mascara more waterproof and smudgeproof. Currently this product application does not exist. Traditional mascara runs and smears. Mascara claiming to be waterproof only lasts 24 hours and can be removed with creams and oils. Tinting, coloring or dyeing the eyelashes with tints or dyes only adds pigment, not volume, texture or length. Tinting, coloring or dyeing the eyelashes is also not permitted in all states. Strip lashes are temporarily glued onto the skin and fall off easily and can not endure any water. Eyelash extensions use an adhesive to adhere one synthetic lash to the existing lash. Eyelash extensions are very costly and take 2 hours to apply.

Cyanoacrylate was invented years ago and has many uses in the medical field. Recently, the specific pharmaceutical grade, black pigmented cyanoacrylate has been used to bond individual eyelash extensions to the eyelashes. However, the pigmented cyanoacrylate has not been used as a stand alone mascara coating application.

Cry Baby Permanent Mascara's combination of microfiber powder polytetrafluoroethylene with the black pigmented cyanoacrylate creates a custom blend with a superior bond to the eyelash. When the coating mixture is applied to the eyelash like traditional mascara, it cures in 2 to 5 minutes, creating a 100% waterproof, smudge and smear proof eyelash coating lasting 2 to 3 weeks.

Consumer mascara applications are not completely waterproof, nor are they completely smudge proof. Physical activities such as swimming or excessive exercise will cause the traditional mascara to run or smear. No other product exists that is completely waterproof and smudgeproof or that lasts 2 to 3 weeks.

### BRIEF SUMMARY OF THE INVENTION

The primary object of the invention is to create a professionally applied semi-permanent mascara that will not run, smear, is 100% waterproof, is antimicrobial, and lasts 2 to 3 weeks.

Another object of the invention is to create a method to professionally apply the semipermanent mascara.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed a composition of matter comprising semi-permanent mascara encompassing a tinted type of pharmaceutical grade cyanoacrylate, mixed with a small amount of polytetrafluoroethene in microfiber powder form, and professionally applied to the eyelashes creating a semi-permanent mascara application that lasts 2 to 3 weeks.

In accordance with a preferred embodiment of the invention, there is disclosed a process to professionally apply a composition of matter comprising semi-permanent mascara which is a tinted type of pharmaceutical grade cyanoacrylate, mixed with a small amount of polytetrafluoroethene in microfiber powder form, creating a semi-permanent mascara application that lasts 2 to 3 weeks.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is a front view of the eye area with bottom eyelashes covered with gel pad.

FIG. 2 is a front view of the eye area with bottom eyelashes not covered by gel pad.

FIG. 3 is a top view of the applicator tools used in the invention.

FIG. 4 is a top view of tools and process to mix the adhesive to the dry powder microfibers.

FIG. 5 is a top view of mixing the adhesive with the dry powder microfibers.

FIG. 6 is a front view of the process to apply the product to top eyelashes.

FIG. 7 is a front view of the process to apply the product to the bottom eyelashes.

FIG. 8 is a front view of the process of precision application of the product to the top eyelashes.

FIG. 9 is a front view of the process and tools used to remove the product from the eyelashes.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

Cry Baby Permanent Mascara is a composition of matter comprising a material and a procedure in which you curl, and coat the eyelashes with waterproof, black pigmented, pharmaceutical grade adhesive coating (a special type of rubber toughened cyanoacrylate). It is mixed with a dry white microfiber powder which consists of an insoluble, irregular shaped polytetrafluoroethylene (PTFE) fibers CAS #9002-84-0. The procedure is to be performed by a certified salon professional.

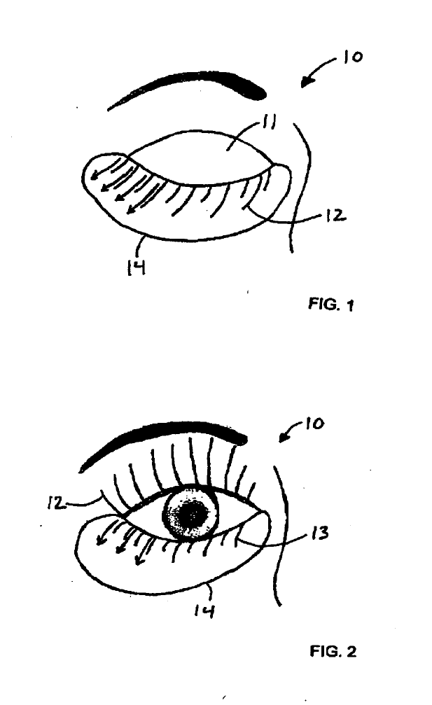

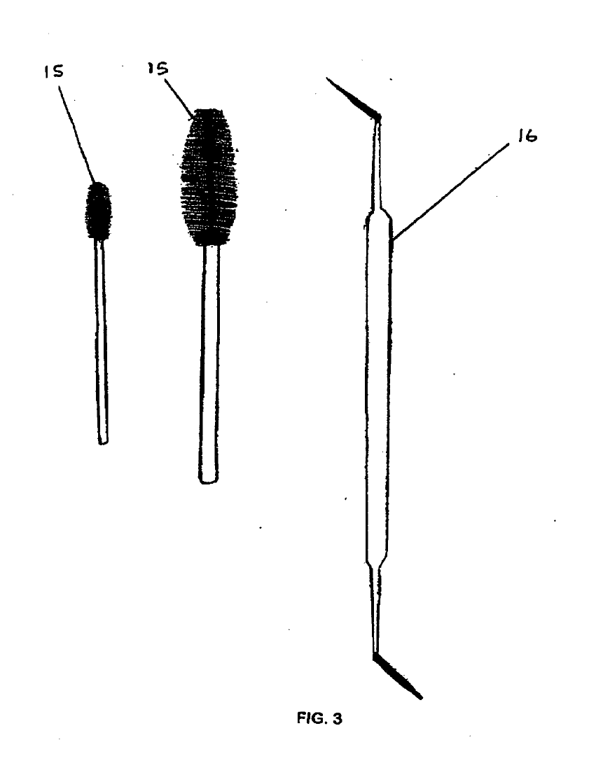

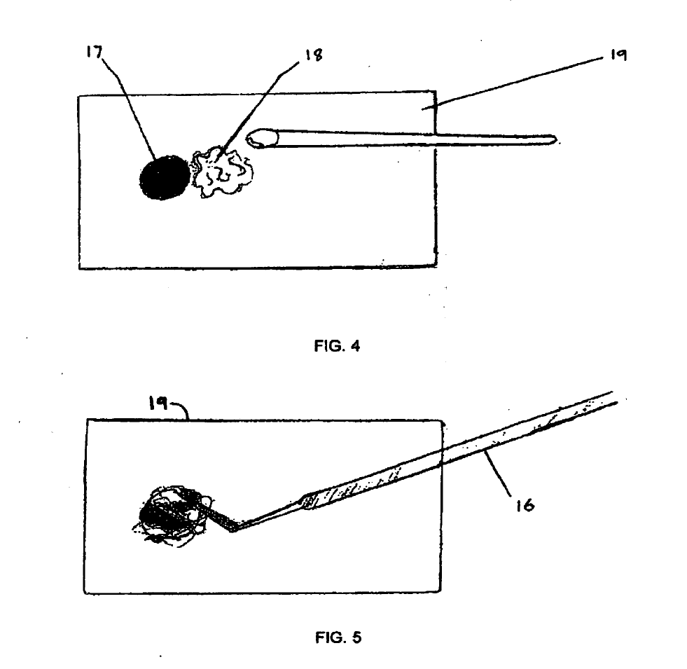

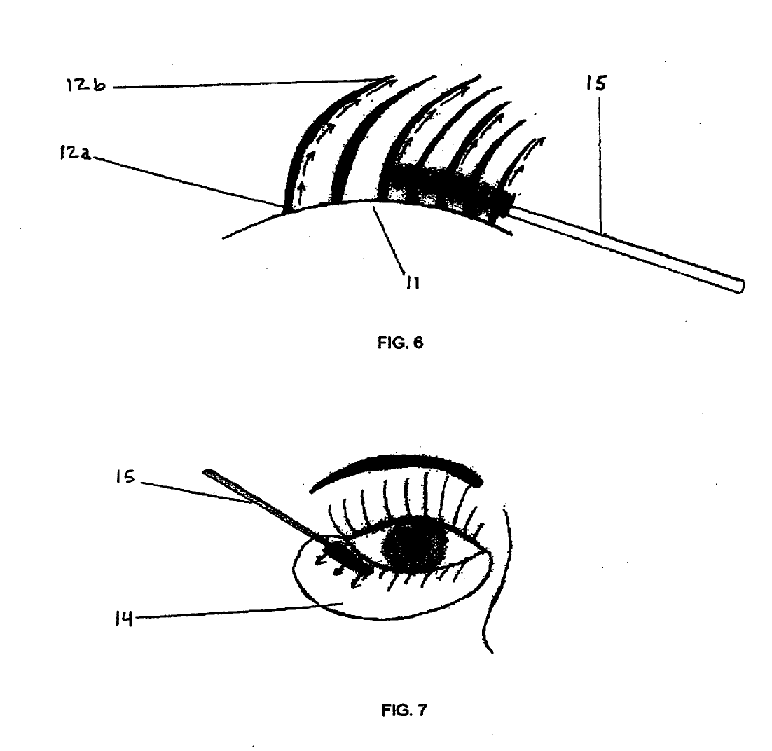

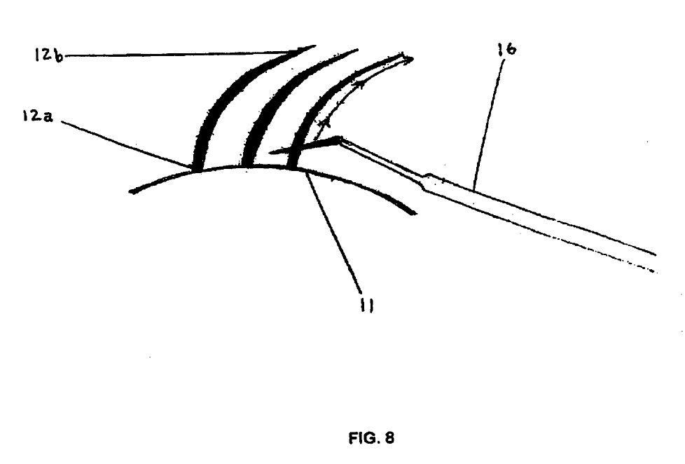

Referring to FIGS. 1-8, the professional first cleans the eye area 10 and places protective medical tape or gel eye pads 14 over the bottom lashes 13 to protect the bottom lash 13 and skin. Next the top lashes 12 are curled with an eyelash wand if necessary.

On a glass slab 19, the adhesive coating (black pigmented cyanoacrylate) 17 is mixed with the dry powder microfibers (PTFE) 18 to the correct consistency (thin to paste like) and is applied with a stainless-steel pick applicator 16 or mascara wand 15 to each individual eyelash 12. It is applied by stroking lashes evenly from lash base 12a to tip 12b and dried with an electric air blower. You repeat until all eyelashes 12 on the top lid 11 are covered and fully dry. The bottom lashes 13 are then cleaned and separated. Medical tape or gel eye pads 14 are placed under the bottom eyelashes 13 to protect the skin. An electric air blower is held on the eye area 10 for client comfort. The bottom lashes 13 are then coated with the adhesive 17 and fiber 18 mixture and dried.

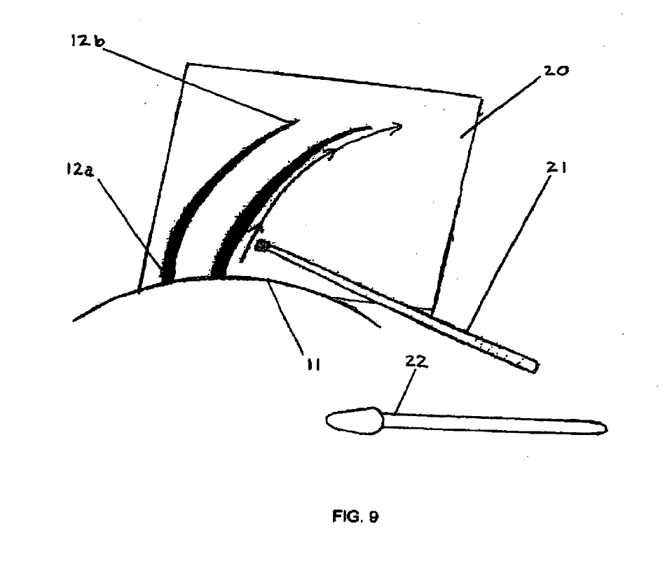

This is a semi-permanent alternative to enhance the natural lash 12, 13 and keep the curl, like traditional mascara, yet it is waterproof, smear proof, and it will never run. An application can last 2-3 weeks or more. The coating can be professionally removed, or it will simply wear off in 2-3 weeks on it's own. Referring to FIG. 9, to remove the coating, the professional holds a non-woven 2×2 pad 20 under or behind the lashes 12, 13. Using a micro brush 21 or foam shadow applicator 22, the professional applies liquid remover by pressing lashes 12, 13 brushing on the remover to release the coating from the lashes 12, 13. Continue until all coating is removed. Rinse lashes 12, 13 with warm water and cleanse.

Client Analysis—

A client with healthy skin and eye area 10.

Anyone with sensitive eyes or skin is not a good candidate.

Materials—

Eyelash curler

Micro fibers (PTFE) 18

Black cyanoacrylate 17

Adhesive remover liquid/gel

Primer (93% alcohol)

Needle nose tweezers

3M-medical tape/Gel eye pads 14

Disposable setup tray

Adhesive applicators/separator

Stainless steel pick 16

Mascara wand 15 (large, medium and mini)

Micro brushes 21

Non-woven 2×2 pads 20

Foam shadow applicators 22

Remover applicator brush/Ultra brush

Electric air blower

Procedure Application—

1. Position client on facial bed or chair.
2. Clean eye and lash area.
3. Apply gel pads 14 or medical tapes to protect bottom lashes 13.
4. Curl top lashes 12 with lash curler (if necessary).
5. Apply primer to the top eyelashes 12.
6. Custom mix adhesive 17 and micro fibers (PTFE) 18 for individual client needs.
7. Separate top lashes 12 and apply adhesive to one top lash 12 at a time or traditionally with a mascara wand 15.
8. Stroke adhesive fiber coating from base 12a to tip 12b and blow dry for 15 seconds.
9. Do not get adhesive coating on the skin or eyelid 11.
10. Repeat until all top lashes 12 are covered and completely dry.
11. Remove bottom lash 13 protector 14.
12. Replace a gel pad 14 or medical tapes under bottom eyelashes 13 to protect skin
13. Clean and prime bottom lashes 13.
14. An electric air blower is used during bottom lash 13 application.
15. Have client hold electric air blower on the eye area 10 on bottom lashes 13.
16. Keep air blowing on eye area 10 the entire application of bottom lashes 13 to insure client comfort.
17. Have client look up with eyes open and begin separating and coating bottom lashes 13.
18. Allow client to blink as necessary.
19. Blow dry lashes 12, 13 thoroughly.

This procedure is for salon professionals only; they must be licensed and certified. The Cry Baby Permanent Mascara procedure should take 30 minutes.

Currently this product application does not exist. Traditional mascara runs and smears and waterproof versions only last 24 hours and can be removed with creams and oils. Tinting, coloring or dyeing the eyelashes with hair color only adds pigment, not volume, texture or length, and it is not allowed in all US states. Strip lashes are temporarily glued onto the skin and fall off easily and cannot endure any water. Eyelash extensions use an adhesive to adhere one synthetic lash to the eyelash and it is very costly and takes 2 hours to apply.

Cry Baby Permanent Mascara is mascara applied by a salon professional that must be trained and certified. It is the texture of traditional mascara but it adheres to the eyelashes and causes a 100% waterproof coating that colors, thickens, lengthens and strengthens the lashes. It allows flexibility with an opaque gloss finish. Once cured it will last 2-3 weeks or longer. There is not a product or application like it. This product application fills a niche. It has the highest performance as a waterproof eyelash coating. It is cost effective and time saving compared to other eyelash extension applications. It is anti-microbial and can wear off on its own without having to be removed at the salon. Once it starts to wear off, one can just start using regular mascara again, or it can be professionally removed and reapplied every 2 to 3 weeks.

Product development—

The adhesive coating or the powdered PTFE microfibers will be developed in different colors using FDA approved pigments for the eye area (i.e. brown, blue, hot pink and purple). Black and clear are the only colors currently available in the adhesive. The pigment will be unique to this procedure only.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. A composition of matter comprising Cry Baby Permanent Mascara
A black pigmented type of pharmaceutical grade cyanoacrylate; mixed with a small amount of irregular shaped, insoluble polytetrafluoroethene in microfiber powder form; and professionally applied to the eyelashes creating a semi-permanent mascara application that lasts 2 to 3 weeks or more.

2. Any type of cyanoacrylate mixed with a small amount of polytetrafluoroethene in microfiber powder form professionally applied to the eyelashes creating a semi-permanent mascara application.

3. A process to professionally apply a composition of matter comprising Cry Baby Permanent Mascara which is a type of black pigmented, pharmaceutical grade cyanoacrylate; mixed with a small amount of irregular shaped, insoluble polytetrafluoroethene in microfiber powder form; creating a semi-permanent mascara application that lasts 2 to 3 weeks.
